SEQUENCE LISTING (8 sequences)

SEQ ID NO: 1 - protein, 22 aa, homo sapiens
EVNQTTLYQRYEIKIVITKMYC

SEQ ID NO: 2 - protein, 15 aa, homo sapiens
CVPPHPQTAFCNSDL

SEQ ID NO: 3 - protein, 14 aa, homo sapiens
KGFQALGDAADIRC

SEQ ID NO: 4 - protein, 21 aa, homo sapiens
RSEEFLIAGKLQDGLLHITTC

SEQ ID NO: 5 - protein, 12 aa, homo sapiens
SEKGFQSRHLAC

SEQ ID NO: 6 - protein, 17 aa, homo sapiens
QTTLYQRYEIKMTKMYK

SEQ ID NO: 7 - protein, 22 aa, homo sapiens
CSFVAPWNSLSLAQRRGFTKTY

SEQ ID NO: 8 - protein, 18 aa, homo sapiens
CLWTDQLLQGSEKGFQSR

(19) United States
(12) Patent Application Publication
(10) Pub. No.: US 2014/0275491 A1
(43) Pub. Date: Sep. 18, 2014

(54) ANTIBODIES RECOGNIZING HUMAN TIMP1 PROTEIN

(71) Applicant: Corgenix Medical Corporation, Broomfield, CO (US)

(72) Inventors: Kelly R. Pitts, Thornton, CO (US); Joseph A. Chapo, Erie, CO (US)

(73) Assignee: Corgenix Medical Corporation, Broomfield, CO (US)

(21) Appl. No.: 14/214,749

(22) Filed: Mar. 15, 2014

Related U.S. Application Data
(60) Provisional application No. 61/798,244, filed on Mar. 15, 2013.

(51) Int. Cl. C07K 16/38

(52) U.S. Cl. CPC C07K 16/38
USPC 530/387.9

(57) ABSTRACT

The present invention is directed to anti-human antibodies recognizing human TIMP1 and the method for making the same. Specifically, the anti-human antibodies are produced using chicken IgY antibodies. The antibodies described herein can be used for the detection and quantification of human TIMP1 protein in patient samples, including bodily fluids, or tissue samples.

### CROSS-REFERENCE TO RELATED APPLICATIONS

This application claims priority to U.S. Provisional Patent Application Ser. No. 61/798,244, filed on Mar. 15, 2013, to Kelly R. Pitts and Joseph A. Chapo entitled “Antibodies Recognizing Human TIMP1 Protein,” currently pending, the entire disclosure of which is incorporated herein by reference.

### BACKGROUND OF THE INVENTION

Commercially available anti-human TIMP1 antibodies have highly variable performance characteristics in several assay formats we have developed. The antibodies of the present application will address several current problems, including (1) lower background and incorrect results due to species specific characteristics, (2) consistent and optimized performance in developed assays, and (3) greater dynamic range from lower background signal.

Numerous commercial sources of anti-human TIMP1 antibodies exist; however, an initial screen of these reagents offers limited positive results in the assays used in the present invention. Known TIMP1 antibodies are as follows:

Anti Human TIMP1 Polyclonal Antibody: Santa Cruz Biotechnology,

Anti Human TIMP1 Poly and Monoclonal Antibody: R and D Systems,

Anti Human TIMP1 Polyclonal Antibody: Millipore/Merck,

Anti Human TIMP1 Poly and Monoclonal Antibody: Abnova Corp.,

Anti Human TIMP1 Polyclonal Antibody: Sigma Aldrich,

Anti Human TIMP1 Poly and Monoclonal Antibody: Acris GmbH,

Anti Human TIMP1 Polyclonal Antibody: Thermo Scientific,

Anti Human TIMP1 Poly and Monoclonal Antibody: Lifespan BioSciences,

Anti Human TIMP1 Polyclonal Antibody: Novus Biologicals, and

Anti Human TIMP1 Polyclonal Antibody: Abeam.

### SUMMARY OF THE INVENTION

The present invention is directed to anti-human antibodies recognizing human TIMP1 and the method for making the same. Specifically, the anti-human antibodies are produced using chicken IgY antibodies. The antibodies described herein can be used for the detection and quantification of human TIMP1 protein in patient samples, including bodily fluids, or tissue samples.

### BRIEF DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a graphic representation showing the results of comparison tests using various chicken IgY antibodies and antibodies known in the art.

### DETAILED DESCRIPTION OF THE INVENTION

The antibodies described herein can be used for the detection and quantification of human TIMP1 protein in patient samples, including bodily fluids, or tissue samples, and the like.

The present invention has, advantages over the previous known art because chickens mount robust immune responses to mammalian immunogens due their evolutionary divergence as compared to common used mammalian species (e.g., rabbits and mice). There is no cross reactivity with mouse, goat or rabbit antibodies. IgY antibodies come from hen's eggs and thus eliminate traditional animal suffering associated with IgG antibody production. Larger quantities of IgY antibodies (whole and subsequent affinity purified) can be generated from a few hens. Once the IgY is purified it is stable for a decade or more at 4-8° C.

FIG. 1 is a graphic representation showing the results of comparison tests using various chicken IgY antibodies and known IgG antibodies. Chicken IgY antibodies are shown in this comparison test as being superior over IgG antibodies.

There is no “Fc domain” within the stem portion of a chicken IgY. This provides several advantages over rabbit IgG's, including that it reduces the likelihood of having false positives in diagnostic applications. Since it is the mammalian Fc domain that binds “rheumatoid factors” or other naturally occurring anti-Fc antibodies, it does not activate mammalian complement systems, allowing the use of chicken IgY's in in vivo applications, and it does not bind to mammalian Fc receptors, avoiding non-specific binding to cells expressing such receptors (e.g., macrophages and dendritic cells). Chicken IgY's contain a larger glycosylation index, allowing more labeling with HRP and other antibody tags, thus produces higher signals.

Chicken polyclonal antibodies produced against the following peptides from human TIMP1 protein sequence: (http://www.nchi.nlm.nih.gov/protein/NP_{—}003245.1)

SEQ ID NO 1:
EVNQTTLYQRYEIKIVITKMYC
SEQ ID NO 2:
CVPPH PQTAFCNSDL
SEQ ID NO 3:
KGFQALGDAADIRC
SEQ ID NO 4:
RSEEFLIAGKLQDGLLHITTC
SEQ ID NO 5:
SEKGFQSRHLAC

Goat polyclonal antibodies produced against the following peptides from human TIMP1 protein sequence: (http://www.ncbi.nlm.nih.gov/protein/NP_{—}003245.1)

SEQ ID NO 6:
QTTLYQRYEIKMTKMYK
SEQ ID NO 7:
CSFVAPWNSLSLAQRRGFTKTY
SEQ ID NO 8:
CLWTDQLLQGSEKGFQSR

### Test Example 1

This test method is designed to determine an antibody's ability to bind its cognate antigen in a sandwich ELISA format. The method relies on either a commercially available ELISA kit or in-house reagents that have been thoroughly tested for detecting purposes. The assay consists of two parts. Part I is the generation of antibody coated microplate (Microplate Preparation). Part II is the actual Hybrid ELISA Microplate Assay.

Procedure: Antibody Screening Assay

Part I—Microplate Preparation

To prepare a microplate for an antibody screening assay for one 96 well microplate use the following calculation:

96(+4 wells)×100 μL/well=100×100 μL=10,000 μL or 10 mL

10 mL(A)×0.002=0.02 mL or 20 μL(B) and 10 mL−20 μL=9.980 mL(C)

Add 100 μL of diluted antibodies to each well of a 96 wellplate. Cover plate with plastic seal and incubate at 4° C. overnight. After 18 hours remove plate from refrigerator and shake off unbound antibody. Add 2004 of Blocking Solution IV to each well and again incubate overnight at 4° C. After 24 hours remove plate from refrigerator and shake out excess Blocking Solution IV. Cover plates with a clean chemical wipe and allow to air dry for at least 18-24 hours. Place plates in clean bag with a desiccant and store at 4° C. till until ready to use.

Part II—Hybrid ELISA Microplate Assay

Prepare dilutions of analyte specific calibrator as outlined below in Table 1. Determine the necessary range of the analyte to be assayed based on the final assay antibodies will be benchmarked against. Make sure the units match (e.g. ng/mL vs. μg/mL, etc.). Dilute Lassa positive control calibrator 1:10.
For example TIMP1-Predicate: ELF Panel (Siemens) Range of assay 3.5-1300 ng/mL
TIMP1 Calibrator Stock: 50 μg/mL (Acro Biosystems)

TABLE 1

| Stock Concentration | Top of Predicate | Dilution |
|---|---|---|
| of Calibrator | Assay Range | Factor |
| (A) 50,000 ng/ML ÷ | (B) 1300 ng/mL = | (C) 38 |
| Example: | (B) 1300 ng/ML = | (C) 38 |
| (A) 50,000 ng/mL ÷ | | |
| Dilution of Calibrator | | |
| Volume of Calibrator | | Volume of Stock |
| to Prepare | Dilution Factor | Calibrator |
| (D) 8000 μL ÷ | (C) 38 = | (E) 210.5 μL |
| Example: | (C) 38 = | (E) 26 μL |
| (D) 1000 μL ÷ | | |
| Volume of Calibrator | Volume of Stock | Volume of Dilution |
| to Prepare | Calibrator | Buffer |
| (D) 1000 μL − | (C) 26 μL = | (E) 974 μL |
| Example: | (C) 26 μL = | (E) 974 μl |
| (D) 1000 μL − | | |

Add (C) to (E) to create Calibrator “1” (1300 ng/mL) Add 500 μL of Calibrator “1” to 500 μL of Calibrator dilution buffer to create Calibrator “2” (650 ng/mL) Add 500 μL of Calibrator “2” to 500 μL of Calibrator dilution buffer to create Calibrator “3” (325 ng/mL) Add 500 μL of Calibrator “3” to 500 μL of Calibrator dilution buffer to create Calibrator “4” (162 ng/mL) Add 500 μL of Calibrator “4” to 500 μL of Calibrator dilution buffer to create Calibrator “5” (81.3 ng/mL) Add 500 μL of Calibrator “5” to 500 μL of Calibrator dilution buffer to create Calibrator “6” (40.6 ng/mL). Add 500 μL of Calibrator “6” to 500 μL of Calibrator dilution buffer to create Calibrator “7” (20.3 ng/mL). Add 1000 μL of Calibrator dilution buffer to create Calibrator “8” (0 ng/mL).

To complete the hybrid ELISA follow the following directions. Add 50 μL of each calibrator to pre-designated wells in the microplate (Note: calibration curve should be done in triplicate). Add 100 μL of the diluted Lassa positive control to control strip wells. 50 μL of the Antibody-HRP Conjugate* test and negative control wells (not to Lassa strip). Add 100 μL of the anti-NP HRP Conjugate to Lassa positive control wells. Cover plate with seal and cover with foil. Incubate with gentle shaking (300 RPM) for 90 minutes at 22-24° C. Remote plate from incubator and shake off reagents. Tap several times to remove any excess. Wash plate 4× with 200 μL of BZN or commercial ELISA Wash Buffer as per manufactures instructions. Tap several times to remove any excess at each wash. Add 100 μL of BioFX TMB substrate to each well. Cover and incubate with gentle shaking for 30 minutes at 22-24° C. Check periodically to make sure reaction is not at saturation. Add 1004 of CGX Stop Solution and read at 450 nm on the plate reader.

The anti-human TIMP1 antibodies described herein function alone or in a combination with the other anti-human TIMP1 antibodies described herein to specifically recognize and bind to human TIMP1 protein. The antibodies described herein can be used to develop a series assay formats including: Enzyme Linked ImmunoSorbent Assay) (ELISA), Lateral Flow Immuno assay (L F I), Immunoturbidimeric Assay (ITA), and Bio chip Assays.

The following pages are non-limiting examples and test procedures for testing the polyclonal antibody of the present invention.

Test Example 2 for testing 4 lots of chicken α-Lu TIMP-1 antibodies according to the following instructions. This test method is designed to determine an antibody's ability to bind its cognate antigen in a sandwich ELISA format. The method relies on either a commercially available ELISA kit or in-house reagents that have been thoroughly tested for detecting purposes. The assay consists of two parts. Part I is the generation of antibody coated microplate (Microplate Preparation). Part II is the actual Hybrid ELISA Microplate Assay. (Note this assay

The following references were used for Example 2:
BULK HABP COATED MICROWELLS (MF01.057)
HABP Coating solution (MF02.045)
Blocking Solution IV (MF02.049)
Nonconformance Reports (SOP1012)
Technical Review Board (SOP1015)
Antibody Capture Lassa ELISA Protocol

The following equipment and materials was used in Example 2:
Sufficient materials from the lot specific packaged kit components to test each control, reference sample and SR sample required for the required number of timepoints.
Analyste specific test antibodies (either PAb or MAb)
Calibrators (e.g. recombinant protein)
BioFX TMB Substrate or substrate from ELISA
CGX Stop Solution
CGX Blocking Solution IV
Maxisorp 96 well μplate Rep Lot 12-0982
1×BZP (PBS) Buffer 20120423D exp 20120323
Calibrator Dilution Buffer
Analyte specific PAb-HRP or Mab-HRP (Ideally from a commercial ELISA that is a predicate or also in-house reagent)* * May need to be titrated based on signal saturation in aassay
Analyte specific ELISA (positive control) or Lassa ELISA
Molecular Dynamics Plate Reader

The following procedure and used for Part I of the antibody screening assay:
1. Dilute all antibodies to be tested to 1 mg/mL (1 μg/μL) in 1×BZP (PBS) buffer.
2. Calculate the amount of antibody required (concentration of Antibody=1 mg/mL) and was used following the below calculations.

| | |
|---|---|
| 2 μg/mL × 100 | 10 mL = 20 |
| 5 | 10 mL = 50 |
| 10 | 10 mL = 100 |
| 20 μg | 10 mL = 200 μg 320 μg |

4/23 Coat Ab plates

21 d: 2 μg/mL 10 mL × C₁V₁ = C₂V₂
2.0 μg/mL (V₁) = (0.002 μg/mL) (10 mL)
V₁ = 0.01 mL = 10 μL

| | |
|---|---|
| 5 μg/mL: | 0.005 μg/mL (10 mL)/2.0 μg/mL = 0.25 mL |
| 10: | 0.010 (10 mL)/2.0 μg/mL = 0.05 mL |
| 20: | 0.020 (10)/2.0 μg/mL = 0.1 mL |

| | | |
|---|---|---|
| 18 d: | 2 | 0.002 (10)/1.5 = 0.015 = 13 μL |
| | 5 | 0.005 (10)/1.5 = 0.033 = 33 μL |
| | 10 | 0.01 (10)/1.5 = 0.06 = 60 μL |
| | 20 | 0.02 (10)/1.5 = 0.03 = 130 μL |
| 19 d: | 2 | 0.002 (10)/2.5 = 0.008 = 8 μL |
| | 5 | 0.005 (10)/2.5 = 0.02 = 20 μL |
| | 10 | 0.010 (10)/2.5 = 0.04 = 40 μL |
| | 20 | 0.020 (10)/2.5 = 0.08 = 80 μL |
| 16 d: | 2 | 0.002 (10)/2.4 = 0.008? = 8.3 μL |
| | 5 | 0.005 (10)/2.4 = 0.208 = 708 μL |
| | 10 | 0.010 (10)/2.4 = 0.0416 = 41.7 μL |
| | 20 | 0.020 (10)/2.4 = 0.083 = 83.3 μL |

3. Add 100 μL of diluted antibodies to each well of a 96 well μplate.
4. Cover plate with plastic seal and incubate @ 4° C. overnight.
5. After 18 hours remove plate from refrigerator and shake off unbound antibody.
6. Add 200 μL of Blocking Solution IV to each well and again incubate overnight at 4° C.
7. After 24 hours remove plate from refrigerator and shake out excess Blocking Solution IV.
8. Cover plates with a clean wipe and allow to air dry for at least 18-24 hours.
9. Plate plates in clean bag with a dessicant and store @ 4° C. till until ready to use.

The following procedure was used for Part II for the hybrid ELISA Microplate Assay:
1. Prepare dilutions of analyte specific calibrator as outlined below:
2. Determine the necessary range of the analyte to be assayed based on the final assay antibodies will be benchmarked against.
3. Dilute Lassa positive control calibrator 1:10. The below table shows what concentrations were used in this Example 2.

| Stock Concentration | Top of Predicate | Dilution |
|---|---|---|
| of Calibrator | Assay Range | Factor |
| (A) 50,000 ng/mL + | (B) 1300 ng/mL = | (C) 38 |
| Example: | (B) 1300 ng/mL = | (C) 38 |
| (A) 50,000 ng/mL + | | |
| Dilution of Calibrator | | |
| Volume of Calibrator | | Volume of Stock |
| to Prepare | Dilution Factor | Calibrator |
| (D) 8000 μL + | (C) 38 = | (E) 210.5 μL/20 |
| | | μL/? |
| Example: | (C) 38 = | (E) 26 μL |
| (D) 1000 μL + | | |
| Volume of Calibrator | Volume of Stock | Volume of Dilution |
| to Prepare | Calibrator | Buffer |
| (D) 1000 μL − | (C) 38 μL = | (E) 26 μL |
| | 26 | 974 |
| Example: | (C) 26 μL = | (D) 974 μL |
| (D) 1000 μL − | | |

4. Add (C) to (E) to create Calibrator “1” (1300 ng/mL)
5. Add 500 μL of Calibrator “1” to 500 μL of Calibrator dilution buffer to create Calibrator “2” (650 ng/mL)
6. Add 500 μL of Calibrator “2” to 500 μL of Calibrator dilution buffer to create Calibrator “3” (32.5 ng/mL)
7. Add 500 μL of Calibrator “3” to 500 μL of Calibrator dilution buffer to create Calibrator “4” (16.2 ng/mL)
8. Add 500 μL of Calibrator “4” to 500 μL of Calibrator dilution buffer to create Calibrator “5” (81.3 ng/mL)
9. Add 500 μL of Calibrator “5” to 500 μL of Calibrator dilution buffer to create Calibrator “6” (40.6 ng/mL)
10. Add 500 μL of Calibrator “6” to 500 μL of Calibrator dilution buffer to create Calibrator “7” (20.3 ng/mL)
11. Add 1000 μL of Calibrator dilution buffer to create Calibrator “8” (0 ng/mL)

The following procedure was used for Part II for the hybrid ELISA:
1. Add 50 μL of each calibrator to pre-designated wells in the microplate (Note: calibration curve should be done in triplicate).
2. Add 100 μL of the dilute Lassa positive control to control strip wells.
3. Add 50 μL of the Antibody-HRP Conjugate* test and negative control wells (not to Lassa strip) * May need to be titrated based on signal saturation in assay
4. Add 100 μL of the anti-NP HRP Conjugate to Lassa positive control wells.
5. Cover plate with seal and cover with foil.
6. Incubate with gentle shaking (300 RPM) for 90 minutes @ 22-24° C.
7. Remove plate from incubator and shake off reagents. Tap several times to remove any excess.
8. Wash plate 4× with 200 μL of BZN or commercial ELISA Wash Buffer as per manufacturer's instructions. Tap several times to remove any excess at each wash.
9. Add 100 μL of BioFX TMB substrate to each well. Cover and incubate with gentle shaking for 5-10 30 minutes @ 22-24° C. Check periodically to make sure reaction is not at saturation.
10. Add 100 μL of CGX Stop Solution and read @ 450 nm on the plate reader.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

From the foregoing it will be seen that this invention is one well adapted to attain all ends and objects hereinabove set forth together with the other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth or shown in the accompanying drawings is to be interpreted as illustrative, and not in a limiting sense.

What is claimed is:

1. A polyclonal antibody directed to an amino acid domain of human TIMP1, wherein the animo acids sequence is set forth in SEQ ID NO:1.

2. A polyclonal antibody directed to an amino acid domain of human TIMP1, wherein the animo acids sequence is set forth in SEQ ID NO:2.

3. A polyclonal antibody directed to an amino acid domain of human TIMP1, wherein the animo acids sequence is set forth in SEQ ID NO:3.

4. A polyclonal antibody directed to an amino acid domain of human TIMP1, wherein the animo acids sequence is set forth in SEQ ID NO:4.

5. A polyclonal antibody directed to an amino acid domain of human TIMP1, wherein the animo acids sequence is set forth in SEQ ID NO:5.

6. A polyclonal antibody directed to an amino acid domain of human TIMP1, wherein the animo acids sequence is set forth in SEQ ID NO:6.

7. A polyclonal antibody directed to an amino acid domain of human TIMP1, wherein the animo acids sequence is set forth in SEQ ID NO:7.

8. A polyclonal antibody directed to an amino acid domain of human TIMP1, wherein the animo acids sequence is set forth in SEQ ID NO:8.
